(19) United States
(12) Patent Application Publication
(10) Pub. No.: US 2015/0257834 A1
(43) Pub. Date: Sep. 17, 2015

(54) MEDICAL GLOVE WITH ANTI-CONTAMINATION POCKET

(71) Applicant: Kamron Mirkarimi, San Diego, CA (US)

(72) Inventor: Kamron Mirkarimi, San Diego, CA (US)

(21) Appl. No.: 14/214,754

(22) Filed: Mar. 15, 2014

(51) Int. Cl. A61B 19/04; A41D 19/00

(52) U.S. Cl. CPC A61B 19/04; A41D 19/0055; A41D 2400/52; A41D 2600/20

(57) ABSTRACT

A disposable medical glove is provided that includes one or more anti-contamination pockets configured to receive a stethoscope diaphragm and thereby protect it from contamination during patient examinations. Additionally, it frees the use of the medical professional's fingers. In a preferred embodiment, the medical glove comprises a glove body, a rectangular pocket at the palmar side of the glove configured to receive the diaphragm, and a rectangular dorsal pocket similarly configured to receive the diaphragm. The free edge of the palmar and dorsal pockets each has a reinforced edge that defines the opening of the respective pocket. In another alternate embodiment, the palmar and/or dorsal pocket each has two compartments (i.e., a primary and second compartment). Alternatively, the glove may have only a single pocket, i.e. a palmar pocket or a dorsal pocket, to suit the user's preference.

### FIELD OF THE INVENTION

The present invention relates to disposable gloves, more particularly medical gloves used by doctors and nurses in the health care industry.

### BACKGROUND OF THE INVENTION

Recent studies have shown that during medical examinations, the stethoscope can retain a relatively large amount of bacteria. While doctors regularly wash and thereby sanitize their hands throughout the day to avoid the spread of germs, little to no attention may be paid to the sanitization of the commonly-used stethoscope. Doctors typically sanitize their hands with a gel or wash after treating a patient, but the stethoscope is simply place back inside the jacket pocket in between uses without sanitizing treatment. This is because sanitizing the diaphragm between uses is impractical. As a result, bacteria from patients are carried on the stethoscope diaphragm and spread to patients. This bacteria can include Methicillin Resistant Staphylococcus Aureus (MRSA), which is highly resistant to antibiotics, and thus not easily sanitized by gels or washes. This form of stethoscope contamination can detract from or altogether negate the anti-contamination benefits provided by hand sanitization. There is a need in the art for a means of minimizing the amount of contamination transferred to and from the stethoscope diaphragm during patient treatments.

### SUMMARY OF THE INVENTION

A disposable medical glove is provided that includes one or more anti-contamination pockets configured to receive a stethoscope diaphragm and thereby protect it from contamination during patient examinations. Additionally, it frees the use of the medical professional's fingers. In a preferred embodiment, the medical glove comprises a glove body, a rectangular pocket on the palmar side of the glove configured to receive the diaphragm, and a rectangular dorsal pocket similarly configured to receive the diaphragm. The free edge of the palmar and dorsal pockets each has a reinforced edge that defines the opening of the respective pocket. In another alternate embodiment, the palmar and/or dorsal pocket each has two compartments (i.e., a primary and second compartment). Alternatively, the glove may have only a single pocket, i.e. a palmar pocket or a dorsal pocket, to suit the user's preference.

### BRIEF DESCRIPTION OF THE DRAWINGS

FIG. 1 illustrates both a frontal and rear view of a disposable medical glove enhanced with a pocket on its palmar and dorsal sides.

FIG. 2 illustrates a perspective view of a disposable medical glove receiving the diaphragm of a stethoscope via a pocket on its palmar side.

FIG. 3 illustrates both a frontal and rear view of an alternate embodiment of a disposable medical glove enhanced with a dual pocket on both its palmar and dorsal sides.

### DETAILED DESCRIPTION

The present invention provides a disposable medical glove is provides that includes one or more anti-contamination pockets configured to receive a stethoscope diaphragm and thereby protect it from contamination during patient examinations.

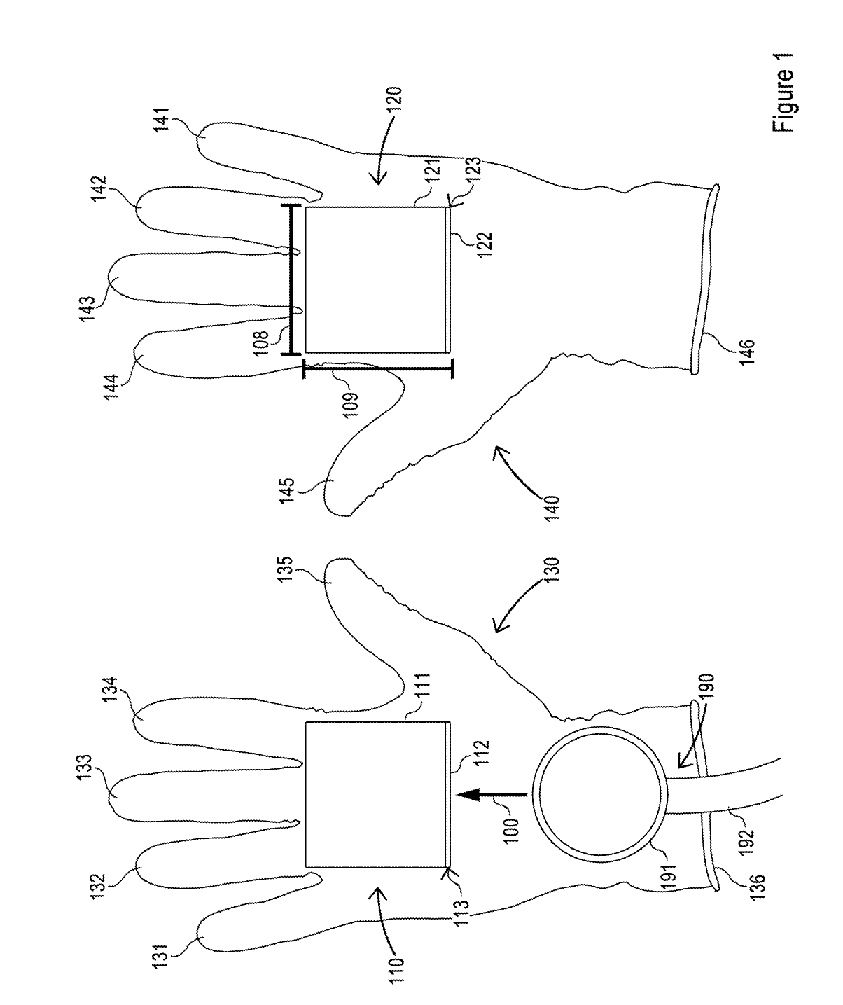

FIG. 1 illustrates both a frontal and rear view of a disposable medical glove enhanced with a rectangular pocket on both its palmar and dorsal sides that are each configured to receive the diaphragm of a stethoscope. The glove's palmar view 130 (left side of FIG. 1) and dorsal view 140 (right side of FIG. 1) are shown in FIG. 1. The glove comprises a glove body 105 (including glove fingers 131-135), palmar pocket 110, and dorsal pocket 120. In a preferred embodiment, the palmar and dorsal pockets are square shaped. The glove body 105 refers to the portion of the glove that resembles a standard glove, i.e. not including the pockets 110 and 120. The palmar and dorsal pockets 110 and 120 are identical in size, shape, and manufacture and can comprised of the same material as the rest of the glove, such as nitrile or latex. Moreover, the pockets occupy a similar position on the glove such that they are superimposed on one another. Referring to the palmar view 130, the palmar pocket 110 comprises an elastic body 111, reinforced edge 113 (i.e. free edge of the pocket), and a pocket opening 112. The elastic body 111 comprises a square section of material whose three perimeter edges are affixed to the glove by methods known in the art. The portion of the palmar pocket 110 that terminates at the opening 112 is lined with a reinforced edge 113 having a reinforced edge or raised rim similar to that found around the hand-receiving opening of a typical disposable glove. Thus, the reinforced edge 113, in conjunction with the glove body 105, forms the pocket opening 112.

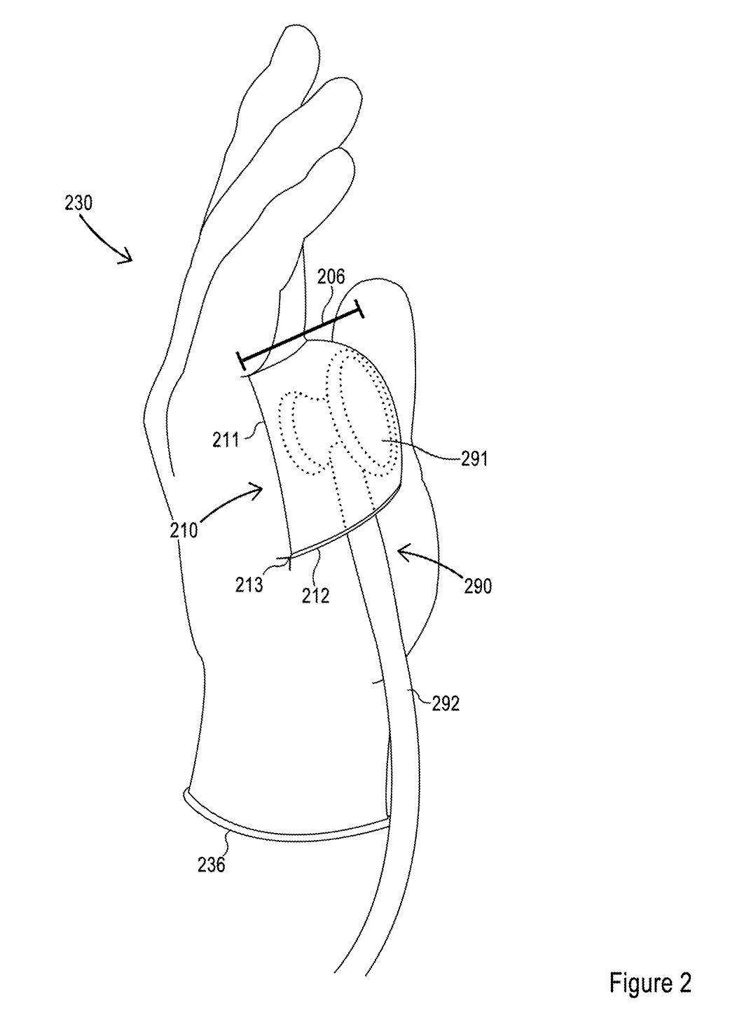

Similarly, with reference to the dorsal view 140 showing the dorsal pocket 120, the elastic body 121 comprises a square section of material whose three perimeter edges are affixed to the glove by methods known in the art. The portion of the palmar pocket 120 that terminates at the opening 122 is lined with a reinforced edge 123. Thus, the reinforced edge 123, in conjunction with the glove body 105, forms the pocket opening 122. As with palmar pocket 110, the dorsal pocket 120 is a square whose width dimension 108 and height dimension 109 are equal. For example, in a preferred embodiment this dimension can be equal to approximately 2.5 inches in order to accommodate a standard stethoscope diaphragm. Similarly, the depth of the pocket may be comfortably opened and elastically stretched to create a depth of approximately 1.5 inches. The depth of the pocket is shown in more detail in FIG. 2 (specifically, the palmar pocket 210). In its unused state (i.e. empty), the pocket ideally possesses minimal depth such that there is minimal slack that could snag or otherwise interfere with use of the glove. In an exemplary embodiment, the glove could have only a single pocket, i.e. only palmar pocket 110 or dorsal pocket 120.

Referring to the reinforced edge 123 of the dorsal pocket 120 as the bottom edge of the pocket (its opposing side being the top, fixed edge), and relative to the glove 140, the pocket's 120 top edge lies on the surface of the glove in a space approximated by (while slightly smaller than) the distance between the index finger 144 and ring finger 142 portions of the glove. It is nearly centered on the vertical axis that bisects the middle finger 143 portion of the glove. The positioning of the pocket maximizes the resulting ergonomics and dexterity of using the stethoscope once inserted into the pocket. The pocket may accept a number of standard medical instruments, and provides sanitary protection by preventing direct contact of the instrument with the patient and thereby preventing contaminants from being transferred onto the instrument and transferred to subsequent patients. Referring back to the palmar view 130, FIG. 1 illustrates how the pocket 110 is especially well-suited to accepting a standard stethoscope 190. As shown by motion arrow 100, a user may insert a stethoscope 190 into the palmar pocket 110 until the stethoscope diaphragm 191 is completely housed within the elastic body 111 of the pocket 110, leaving the rubber tubing 192 outside of the pocket. This inserted state is better visualized in FIG. 2.

FIG. 2 illustrates a perspective view of a disposable medical glove including a palmar pocket 210 on its palmar side, with a stethoscope inserted into the pocket. With the stethoscope diaphragm held in the pocket, two advantages are provided. First, the medical professional's fingers are free for other uses. Second, and more critically, the stethoscope diaphragm is covered by the pocket and does not contact the patient during examination. Thus, contaminants such as germs and bacteria are not transferred to the diaphragm and thereafter between patients. This is particularly beneficial because the diaphragm does not need to be sanitized between uses (which is not common practice anyway due to the inconvenience and lack of time).

The glove comprises a glove body 205 (including glove fingers 231-135), palmar pocket 210, and dorsal pocket 220. Here, the diaphragm 291 of the stethoscope 290 has been fully inserted into the elastic body 211 of the pocket 210. While FIG. 2 illustrates the insertion of the stethoscope diaphragm into the palmar pocket, the same applies to the dorsal pocket. The stethoscope is inserted into the pocket 210 such a way that the elastic body 211 comfortably houses the diaphragm 291, thereby covering the face of the diaphragm with a layer of elastic material which then makes contact with the patient. The lack of direct contact between the diaphragm and the patient ensures that contaminants (e.g. germs or bacteria) are not transferred between the diaphragm and the patient. Once the diaphragm is inserted into the pocket, only the rubber tubing 292 extends exits the pocket 210, becoming visible at the exit of the singular opening 212, below the reinforced edge 213. The depth of the pocket 210 is seen here near or at the point of optimal elastic stretching (one and one half inches), which is indicated by depth dimension 206. The pocket should be configured so that there is enough slack so that the diaphragm can be easily inserted, but so much that it results in unwanted snagging. The elastic body 211 has a composition that provides an appropriate level of elasticity for pocket expansion in this capacity, additionally holding the stethoscope's diaphragm in place with adequate tension. This tension can be adjusted based on the thickness and type of plastic/polymer use for the pocket.

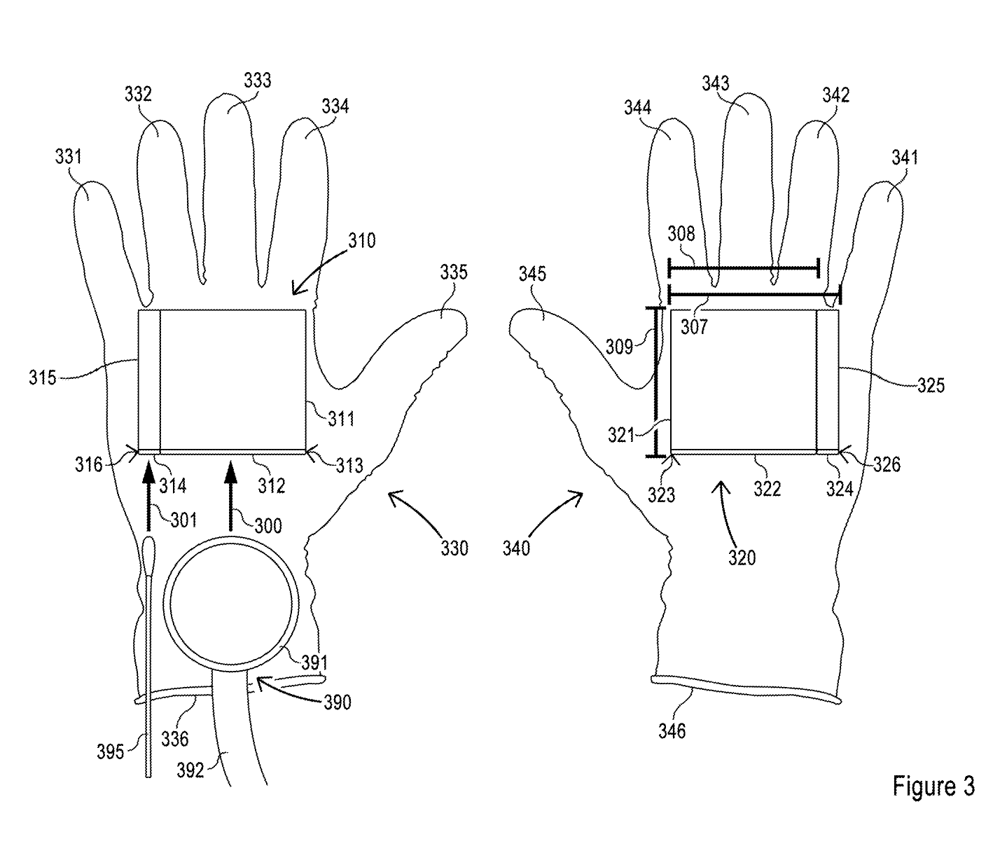

FIG. 3 illustrates both a frontal and rear view of an alternate embodiment of a disposable medical glove enhanced with a dual pocket on both its palmar and dorsal sides. As with the previous embodiment, the attached dual pocket exists with the same form and function on either side of the glove (planar and dorsal). Similarly, the glove comprises a glove body 305 (including glove fingers 331-135), palmar pocket 310, and dorsal pocket 320. Referring to the palmar view 330, the dual pocket 310 comprises a primary elastic body (primary pocket) 311, a secondary elastic body (secondary pocket) 315, a primary opening 312, and a secondary opening 314. The primary opening 312 is lined with a primary raised rim 313. Likewise, the secondary opening 314 is lined with a secondary raised rim 316. Comparatively speaking (with regard to the previous embodiment), in describing the general configuration of the dual pocket on the surface of the medical glove, it can be safely assumed that the entire dual pocket must be situated slightly lower (i.e. further from the tips of the glove fingers 331-334, 341-344) than the single pocket in the previous embodiment. The reason for this is that its wider extended width dimension 307 would otherwise interfere with the proximal base of the pinky finger 331, 341 portion of the medical glove.

Referring now to the dorsal view 340, the dual pocket 320 is a rectangular-shaped, its width represented by extended width dimension 307 and its height represented by height dimension 309. Along with its height dimension, the width of its primary elastic body 321 remains the same as in the previous embodiment, as denoted by width dimension 308. The spirit of the invention's functionality remains basically consistent between embodiments, this embodiment simply elaborates on the initial embodiment's functionality by exploring the possibility of an additional smaller adjacent pocket (i.e. elastic body 315, 325) that is particularly suited to hold a medical swab or similarly sized object in order to provide additional convenience or contamination prevention. As illustrated in FIG. 3, a stethoscope 390 may be inserted into the dual pocket 310 via motion arrow 300, whereby its diaphragm 391 is fully housed within the primary elastic body 311, and its rubber tubing 392 extends out of the primary opening 312. Likewise, and unique to this embodiment, a medical swab 395 may be inserted into the dual pocket 310 via motion arrow 301, whereby the majority of its form is housed within the secondary elastic body 315. The swab may also be placed into the secondary elastic body whilst in its given sterile packaging, package size permitting. This methodology provides a more convenient method of temporary storage for the swab during a patient's office visit, making it an asset in the highly time-sensitive realm of medical treatment.

While there have been described herein what are considered to be preferred and exemplary embodiments of the present invention, other modifications of the invention shall be apparent to those skilled in the art from the teachings herein. For example, the relative dimensions of the device may be altered while keeping within the spirit and teachings of the invention. Moreover, the glove pocket can be configured to receive an object or instrument other than a stethoscope diaphragm, as is appropriate for the desired application. It is therefore desired to be secured, in the appended claims all such modifications as fall within the spirit and scope of the invention.

What is claimed is:

1. A disposable glove comprising:
a glove body having a palmar side and a dorsal side;
a rectangular palmar pocket on the palmar side of the glove, said palmar pocket having a primary and secondary compartment;
wherein the primary compartment is configured to receive a stethoscope diaphragm and the secondary compartment is configured to receive a medical swab;
wherein the palmar pocket comprises a fixed end and a free end, said free end having a reinforced edge that defines the opening of the pocket.
a rectangular dorsal pocket on the dorsal side of the glove, said dorsal pocket having a primary and secondary compartment;
wherein the primary compartment is configured to receive a stethoscope diaphragm and the secondary compartment is configured to receive a medical swab; and
wherein the dorsal pocket comprises a fixed end and a free end, said free end having a reinforced edge that defines the opening of the pocket.

2. The disposable glove of claim 1 that is composed of nitrile or latex.

3. A disposable glove comprising:
a glove body having a palmar side and a dorsal side;
a rectangular palmar pocket on the palmar side of the glove, said palmar pocket configured receive a stethoscope diaphragm;
wherein the palmar pocket comprises a fixed end and a free end, said free end having a reinforced edge that defines the opening of the pocket.
a rectangular dorsal pocket on the dorsal side of the glove, said dorsal pocket configured to receive a stethoscope diaphragm; and
wherein the dorsal pocket comprises a fixed end and an free end, said free end having a reinforced edge that defines the opening of the pocket.

4. The disposable glove of claim 3 that is composed of nitrile or latex.

5. A disposable glove comprising:
a glove body having a palmar side and a dorsal side;
a rectangular pocket on either the palmar side or dorsal side of the glove, said pocket configured to receive a stethoscope diaphragm; and
wherein the pocket comprises a fixed end and an free end, said free end having a reinforced edge that defines the opening of the pocket.

6. The disposable glove of claim 5 that is composed of nitrite or latex.
